(19) United States
(12) Patent Application Publication
(10) Pub. No.: US 2017/0100315 A1
(43) Pub. Date: Apr. 13, 2017

(54) SUNSCREEN COMPOSITION

(71) Applicant: Henkel AG & Co. KGaA, Duesseldorf (DE)

(72) Inventors: Susanne Dickhof, Viersen (DE); Marianne Waldmann-Laue, Monheim (DE); Soraya Heinen, Koeln (DE); Ira Hartmann, Duesseldorf (DE)

(73) Assignee: Henkel AG & Co. KGaA, Duesseldorf (DE)

(21) Appl. No.: 15/288,090

(22) Filed: Oct. 7, 2016

(30) Foreign Application Priority Data
Oct. 12, 2015 (DE) 10 2015 219 715.8

(51) Int. Cl. A61K 8/37; A61Q 17/04; A61K 8/34; A61K 8/44; A61K 8/49; A61K 8/35; A61K 8/40; A61K 8/06; A61K 8/31

(52) U.S. Cl. CPC A61K 8/375; A61K 8/062; A61Q 17/04; A61K 8/342; A61K 8/31; A61K 8/37; A61K 8/4946; A61K 8/35; A61K 8/40; A61K 8/44

(57) ABSTRACT

Cosmetic compositions in the form of an oil-in-water emulsion include:
a) at least one monoglycerol fatty acid ester A,
b) at least one first polyglycerol difatty acid ester B,
c) at least one second polyglycerol difatty acid ester C which differs from the first polyglycerol difatty acid ester B,
d) at least one fatty acid ester of the general formula (I)

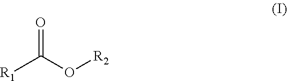

the residues R₁ and R₂ mutually independently denoting a saturated or unsaturated alkyl residue with 16 to 26 carbon atoms, and
e) at least one organic UV filter.

Methods of using such a composition is also disclosed.

### FIELD OF THE INVENTION

The present invention generally relates to cosmetic compositions for protecting skin and hair from UV radiation and to a method using such cosmetic compositions.

### BACKGROUND OF THE INVENTION

Human skin, the body's largest organ, performs numerous vital functions. Having a surface area of average approximately 2 m² in adults, the skin has a very major role as a protective and sense organ. The task of this organ is to mediate and provide defense against mechanical, thermal, actinic, chemical and biological stimuli. The skin accordingly also protects the human body inter alia from the harmful action of the ultraviolet part of solar radiation, but despite its own protective mechanisms is itself also damaged by this ultraviolet radiation.

Excessive UV-B radiation (radiation in the range between 290 nm and 320 nm) may, for example, lead to erythema, simple sunburn or indeed to skin burns of greater or less severity. Sunlight has its maximum erythemic action in the range around 308 nm.

While it was originally assumed that the longer-wave UV-A radiation with a wavelength of between 320 nm and 400 nm had only a negligible biological action, numerous studies have now demonstrated that UV-A radiation is far more dangerous than UV-B radiation with regard to triggering photodynamic, specifically phototoxic responses and chronic changes in the skin.

Furthermore, the harmful influence of UV-B radiation may be further boosted by UV-A radiation.

For instance, it has been found that even UV-A radiation under entirely normal everyday conditions is sufficient to damage the collagen and elastin fibres, which are of great significance to the structure and strength of the skin, within a short time. This results in chronic light-dependent skin changes, the skin “ageing” prematurely. Clinical manifestations of light-aged skin include, for example, lines and wrinkles and an irregular, furrowed relief. Areas of skin affected by light-dependent skin ageing may also exhibit irregular pigmentation. Brown spots, keratoses and even carcinomas or malignant melanomas may also form.

Skin prematurely aged by everyday UV exposure is moreover distinguished by lower activity of the Langerhans cells and chronic, low-level inflammation.

Some 90% of the ultraviolet radiation reaching the earth consists of UV-A rays. While UV-B radiation varies greatly as a function of numerous factors (for example season, time of day or latitude), UV-A radiation remains relatively constant irrespective of factors governed by season, time of day or geography. At the same time, the majority of UV-A radiation penetrates into the living epidermis, whereas approximately 70% of the UV-B rays are absorbed by the stratum corneum.

Cosmetic photoprotective compositions are used to protect the skin from the harmful effects of UV radiation. These cosmetic photoprotective compositions include at least one, but generally a combination of UV filter substances.

The UV filter substances are substances which assume liquid or crystalline form at room temperature and which are capable of absorbing ultraviolet rays and releasing the absorbed energy again in the form of longer-wave radiation, for example heat. A distinction is drawn between UV-A filters and UV-B filters, depending on the wavelength range in which the absorption maximum of the filter is located.

It is often very difficult to incorporate organic UV filters into cosmetic carriers in the quantities required for a high sun protection factor in such a manner that compositions which have long-term stability and pleasant, in particular non-tacky, properties are obtained.

The problem addressed by the present invention was accordingly that of providing photoprotective compositions which include at least one organic UV filter, assume storage-stable form, thus are both chemically and physically stable, and are distinguished by good cosmetic properties, in particular low tackiness and good skin compatibility.

German patent application DE 199 19 630 Al describes sunscreen preparations which, in addition to further components, include polyglycerol fatty acid esters and organic UV filters.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic composition in the form of an oil-in-water emulsion includes at least one monoglycerol fatty acid ester A, at least one first polyglycerol difatty acid ester B, at least one second polyglycerol difatty acid ester C which differs from the first polyglycerol difatty acid ester B, at least one fatty acid ester of the general formula (I)

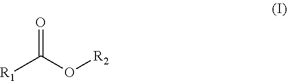

the residues R₁ and R₂ mutually independently denoting a saturated or unsaturated alkyl residue with 16 to 26 carbon atoms, and at least one organic UV filter.

A combination that includes at least one monoglycerol fatty acid ester A, at least one first polyglycerol difatty acid ester B, at least one second polyglycerol difatty acid ester C which differs from the first polyglycerol difatty acid ester, and at least one fatty acid ester of the general formula (I)

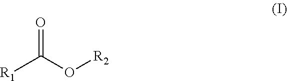

the residues R₁ and R₂ mutually independently denoting a saturated or unsaturated alkyl residue with 16 to 26 carbon atoms, is used for improving the storage stability of a cosmetic composition including at least one UV filter; improving the UV-protective action of a cosmetic composition including at least one UV filter; improving the absorbability of a cosmetic composition including at least one UV filter; and improving the spreadability and reducing the tackiness of a cosmetic composition including at least one UV filter.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that cosmetic photoprotective compositions which, in addition to an organic UV filter, furthermore include a combination of four specific fatty acid esters solve the above-stated problems. Cosmetic compositions on the basis of these combinations are distinguished from conventional prior art compositions by good cosmetic compatibility, good application properties and low tackiness and are furthermore also physically and chemically stable at elevated temperatures over an extended period.

Physical stability here denotes the stability of the rheological properties of the cosmetic composition and the stability thereof against phase separation, including stability against settling of suspended solids.

Chemical stability denotes stability against chemical transformation or decomposition, as may be verified for example by activity testing of the cosmetically active substances d).

The present invention provides:
1. A cosmetic composition in the form of an oil-in-water emulsion including:
   a) at least one monoglycerol fatty acid ester A,
   b) at least one first polyglycerol difatty acid ester B,
   c) at least one second polyglycerol difatty acid ester C which differs from the first polyglycerol difatty acid ester B,
   d) at least one fatty acid ester of the general formula (I)

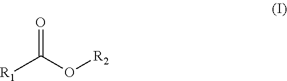

the residues R₁and R₂ mutually independently denoting a saturated or unsaturated alkyl residue with 16 to 26 carbon atoms,
e) at least one organic UV filter.
2. A cosmetic composition according to point 1, wherein the monoglycerol fatty acid ester A is selected from the group of mono-, di- or triesters of glycerol with behenic acid.
3. A cosmetic composition according to one of the preceding points, wherein the monoglycerol fatty acid ester A is selected from the group of compounds with the INCI name Glyceryl Behenate.
4. A cosmetic composition according to one of the preceding points, wherein the monoglycerol fatty acid ester A is present, relative to the total weight of the compositions, in quantities of 0.01 to 6.0 wt. %, preferably in quantities of 0.1 to 4.0 wt. % and in particular in quantities of 0.5 to 2.0 wt. %.
5. A cosmetic composition according to one of the preceding points, wherein the first polyglycerol difatty acid ester B is selected from the group of di-, tri- or tetraglycerol difatty acid esters, preferably from the group of triglycerol difatty acid esters.
6. A cosmetic composition according to one of the preceding points, wherein the first polyglycerol difatty acid ester B is selected from the group of triglycerol difatty acid esters of saturated or unsaturated fatty acids with 16 to 30 carbon atoms, preferably from the group of triglycerol difatty acid esters of fatty acids from the group palmitic acid, palmitoleic acid, oleic acid, cerotic acid and melissic acid.
7. A cosmetic composition according to one of the preceding points, wherein the first polyglycerol difatty acid ester B is selected from the group of compounds with the INCI name PEG-3 Beeswax.
8. A cosmetic composition according to one of the preceding points, wherein the first polyglycerol difatty acid ester B is present, relative to the total weight of the compositions, in quantities of 0.05 to 3.0 wt. %, preferably in quantities of 0.1 to 2.0 wt. % and in particular in quantities of 0.2 to 1.5 wt. %.
9. A cosmetic composition according to one of the preceding points, wherein the second polyglycerol difatty acid ester C is selected from the group of penta-, hexa- or heptaglycerol difatty acid esters, preferably from the group of hexaglycerol difatty acid esters.
10. A cosmetic composition according to one of the preceding points, wherein the second polyglycerol difatty acid ester C is selected from the group of hexaglycerol difatty acid esters of saturated or unsaturated fatty acids with 16 to 30 carbon atoms, preferably from the group of hexaglycerol difatty acid esters of saturated or unsaturated fatty acids with 16 to 24 carbon atoms and in particular from the group of hexaglycerol difatty acid esters of stearic acid.
11. A cosmetic composition according to one of the preceding points, wherein the second polyglycerol difatty acid ester C is selected from the group of compounds with the INCI name PEG-6 Distearate.
12. A cosmetic composition according to one of the preceding points, wherein the second polyglycerol difatty acid ester C is present, relative to the total weight of the compositions, in quantities of 0.2 to 8.0 wt. %, preferably of 0.4 to 6.0 wt. % and in particular of 0.6 to 4.0 wt. %.
13. A cosmetic composition according to one of the preceding points, wherein in the general formula (I) the residues R₁ and R₂ mutually independently denote an unsaturated alkyl residue with 16 to 26 carbon atoms, preferably an unsaturated alkyl residue with 18 to 24 carbon atoms.
14. A cosmetic composition according to one of the preceding points, wherein the fatty acid ester of the general formula (I) is present, relative to the total weight of the compositions, in quantities of 0.05 to 3.0 wt. %, preferably in quantities of 0.1 to 2.0 wt. % and in particular in quantities of 0.2 to 1.5 wt. %.
15. A cosmetic composition according to one of the preceding points, wherein the organic UV filter is selected from the group of substituted dibenzoylmethanes, diphenylacrylic acid esters, salicylic acid esters and benzimidazoles.
16. A cosmetic composition according to one of the preceding points, wherein 3,3,5-trimethylcyclohexyl salicylate is used as the organic UV filter.
17. A cosmetic composition according to one of the preceding points, wherein 2-phenylbenzimidazole-5-sulfonic acid and the salts thereof is used as the organic UV filter.
18. A cosmetic composition according to one of the preceding points, wherein 1-(4-tert.-butylphenyl)-3-(4-methoxyphenyl)propane-1,3-dione is used as the organic UV filter.
19. A cosmetic composition according to one of the preceding points, wherein 1,1′ -diphenylacrylonitrile acid 2-ethylhexyl ester, preferably in combination with ethylenediaminetetraacetic acid and the salts thereof, is used as the organic UV filter.
20. A cosmetic composition according to one of the preceding points, wherein phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine, is used as the organic UV filter.
21. A cosmetic composition according to one of the preceding points, wherein the organic UV filter includes at least two, preferably at least three and in particular at least four organic UV filters from the group 3,3,5-trimethylcyclohexyl salicylate, 2-phenylbenzimidazole-5-sulfonic acid and the salts thereof, 1-(4-tert.-butylphenyl)-3-(4-methoxyphenyl)propane-1,3-dione, 1,1′-diphenylacrylonitrile acid 2-ethylhexyl ester, and phenylene-1,4-bis-(2-benzimidazole)-3,3′-5,5′-tetrasulfonic acid and the salts thereof.
22. A cosmetic composition according to one of the preceding points, wherein the UV filters are present, relative to the total weight of the compositions, in quantities of 1.0 to 35 wt. %, preferably of 2.0 to 30 wt. % and in particular of 5.0 to 25 wt. %.
23. A cosmetic composition according to one of the preceding points, wherein the composition furthermore includes
   f) a linear fatty alcohol, preferably a linear fatty alcohol with 14 to 18 carbon atoms and in particular a linear fatty alcohol with 16 carbon atoms.
24. A cosmetic composition according to point 23, wherein the linear fatty alcohol is present, relative to the total weight of the compositions, in quantities of 0.01 to 5.0 wt. %, preferably in quantities of 0.05 to 3.0 wt. % and in particular in quantities of 0.1 to 1.0 wt. %.
25. A cosmetic composition according to one of the preceding points, wherein the composition furthermore includes
   g) a linear alkane with 11 to 18 carbon atoms, preferably a linear alkane with 11 to 13 carbon atoms and in particular a linear alkane with 11 and/or 13 carbon atoms.
26. A cosmetic composition according to point 25, wherein the linear alkane is present, relative to the total weight of the compositions, in quantities of 0.1 to 10 wt. %, preferably of 0.2 to 7.0 wt. % and in particular of 0.5 to 5.0 wt. %.
27. A cosmetic composition according to one of the preceding points, wherein the composition furthermore includes at least one hydrophobically modified (meth)acrylic acid copolymer.
28. A cosmetic composition according to point 27, wherein the hydrophobically modified (meth)acrylic acid copolymer is attributable to
   at least one monomer from the group acrylic acid, methacrylic acid, C₁-C₆ alkyl acrylic acid esters or C₁-C₆ alkyl methacrylic acid esters, and
   at least one monomer from the group of C₁₀₋₃₀ alkyl acrylates, C₁₀₋₃₀ alkyl methacrylates, C₁₀₋₃₀ alkyl PEG acrylates, C₁₀₋₃₀ alkyl PEG methacrylates or C₁₀₋₃₀ alkyl PEG itaconates, preferably from the group of C₁₀₋₃₀ alkyl acrylates.
29. A cosmetic composition according to one of points 27 or 28, wherein the hydrophobically modified (meth)acrylic acid copolymer is present in quantities of 0.05 to 2.0 wt. %, preferably of 0.1 to 1.2 wt. % and in particular of 0.2 to 0.8 wt. %.
30. A cosmetic composition according to one of the preceding points, wherein the composition furthermore includes at least one copolymer of 2-methyl-2[(1-oxo-2-propenyl)amino]-1-propanesulfonic acid.
31. A cosmetic composition according to point 30, wherein the copolymer of 2-methyl-2[(1-oxo-2-propenyl)amino]-1-propanesulfonic acid is attributable to
   2-methyl-2[(1-oxo-2-propenyl)amino]-1-propanesulfonic acid and
   vinylpyrrolidone.
32. A cosmetic composition according to one of points 30 or 31, wherein the copolymer of 2-methyl-2 [(1-oxo-2-propenyl)amino]-1-propane sulfonic acid is present, relative to the total weight of the composition, in quantities of 0.05 to 3.0 wt. %, preferably of 0.1 to 2.0 wt. % and in particular of 0.2 to 1.5 wt. %.
33. Use of a combination comprising
   a) at least one monoglycerol fatty acid ester A,
   b) at least one first polyglycerol difatty acid ester B,
   c) at least one second polyglycerol difatty acid ester C which differs from the first polyglycerol difatty acid ester,
   d) at least one fatty acid ester of the general formula (I)

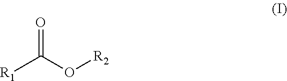

the residues R₁ and R₂ mutually independently denoting a saturated or unsaturated alkyl residue with 16 to 26 carbon atoms, for
improving the storage stability of a cosmetic composition including at least one UV filter;
improving the UV-protective action of a cosmetic composition including at least one UV filter;
improving the absorbability of a cosmetic composition including at least one UV filter;
improving the spreadability and reducing the tackiness of a cosmetic composition including at least one UV filter.
34. A method for cosmetic skin care, in which a cosmetic composition according to one of points 1 to 32 is applied onto the skin.

The present invention firstly provides a cosmetic composition in the form of an oil-in-water emulsion including:
a) at least one monoglycerol fatty acid ester A,
b) at least one first polyglycerol difatty acid ester B,
c) at least one second polyglycerol difatty acid ester C which differs from the first polyglycerol difatty acid ester,
d) at least one fatty acid ester of the general formula (I)

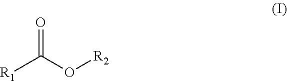

the residues R₁ and R₂ mutually independently denoting a saturated or unsaturated alkyl residue with 16 to 26 carbon atoms, and
e) at least one organic UV filter.

The cosmetic compositions according to the invention assume the form of an oil-in-water emulsion and thus include an aqueous fraction. The composition according to the invention is preferably viscous-liquid. Preferred cosmetic compositions are characterized by a water content, relative to the total weight thereof, of 30 to 82 wt. %, preferably 35 to 75 wt. %, in particular 45 to 60 wt. %.

In addition to water, the compositions according to the invention may furthermore contain, relative to the total weight thereof, 1 to 15 wt. %, preferably 2 to 12 and in particular 3 to 10 wt. % of at least one alcohol. Preferred alcohols are glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycols, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, polyethylene glycol and mixtures of the above-stated substances. The compositions according to the invention more preferably include glycerol as the alcohol.

The cosmetic compositions include at least one monoglycerol fatty acid ester A as the first essential component thereof.

Suitable fatty acids are in particular C₁₆₋₂₂ carboxylic acids, preferably saturated, linear C₁₆₋₂₂ carboxylic acids. The monoglycerol fatty acid ester A is more preferentially selected from the group of mono-, di- or triesters of glycerol with behenic acid. One exemplary monoglycerol fatty acid ester A which is particularly advantageous with regard to the action thereof is a mixture of mono-, di- and triglycerides of behenic acid. Mixtures including 10 to 25 wt. % monoglyceryl behenate, 40 to 65 wt. % diglyceryl behenate and 25 to 50 wt. % triglyceryl behenate are particularly preferred.

The monoglycerol fatty acid ester A is more preferentially selected from the group of compounds with the INCI name Glyceryl Behenate. One suitable ester is obtainable under the trade name Compritol® 888 CG (from Gattefosse).

The proportion by weight of the monoglycerol fatty acid ester A in the total weight of cosmetic compositions preferably amounts to from 0.01 to 6.0 wt. %, preferably from 0.1 to 4.0 wt. % and in particular from 0.5 to 2.0 wt. %.

The polyglycerol difatty acid ester B is a further essential component of the cosmetic compositions.

Examples of polyglycerol difatty acid esters B are Polyglyceryl-2 Diisostearate, Polyglyceryl-2 Dioleate, Polyglyceryl-2 Dipolyhydroxystearate, Polyglyceryl-2 Distearate, Polyglyceryl-3 Dicaprate, Polyglyceryl-3 Dicocoate, Polyglyceryl-3 Di-Hydroxystearate, Polyglyceryl-3 Diisostearate, Polyglyceryl-3 Dioleate or Polyglyceryl-3 Distearate.

Preferred polyglycerol difatty acid esters B are selected from the group of di-, tri- or tetraglycerol difatty acid esters, preferably from the group of triglycerol difatty acid esters.

Thanks to the cosmetic and technical action thereof, preferred polyglycerol difatty acid esters B are those from the group of triglycerol difatty acid esters of saturated or unsaturated fatty acids with 16 to 30 carbon atoms, preferably from the group of triglycerol difatty acid esters of fatty acids from the group palmitic acid, palmitoleic acid, oleic acid, cerotic acid and melissic acid.

Examples of polyglycerol difatty acid esters B are Polyglyceryl-2 Dipalmitate, Polyglyceryl-2 Dipalmitoleate, Polyglyceryl-2 Dioleate, Polyglyceryl-2 Dicerotinate, Polyglyceryl-2 Dimelissinate, Polyglyceryl-3 Dipalmitate, Polyglyceryl-3 Dipalmitoleate, Polyglyceryl-3 Dioleate, Polyglyceryl-3 Dicerotinate, Polyglyceryl-3 Dimelissinate, Polyglyceryl-4 Dipalmitate, Polyglyceryl-4 Dipalmitoleate, Polyglyceryl-4 Dioleate, Polyglyceryl-4 Dicerotinate and Polyglyceryl-4 Dimelissinate. Polyglyceryl-3 Dipalmitate, Polyglyceryl-3 Dicerotinate and Polyglyceryl-3 Dimelissinate are more preferred.

Palmitic acid, palmitoleic acid, oleic acid, cerotic acid and melissic acid are components of beeswax and the above-stated polyglycerol difatty acid esters can be produced inter alia by reacting polyglycerol with the free acids of beeswax. More preferred polyglycerol difatty acid esters B are selected from the group of compounds with the INCI name PEG-3 Beeswax.

The proportion by weight of the polyglycerol difatty acid ester B in the total weight of the cosmetic compositions is preferably from 0.05 to 3.0 wt. %, preferably from 0.1 to 2.0 wt. % and in particular from 0.2 to 1.5 wt. %.

The cosmetic compositions include a second polyglycerol difatty acid ester C which differs from the first polyglycerol difatty acid ester B as the third essential component thereof.

Examples of polyglycerol difatty acid esters C are Polyglyceryl-4 Dilaurate, Polyglyceryl-5 Dilaurate, Polyglyceryl-5 Dioleate, Polyglyceryl-6 Dicaprate, Polyglyceryl-6 Diisostearate, Polyglyceryl-6 Dioleate, Polyglyceryl-6 Dipalmitate, Polyglyceryl-6 Distearate, Polyglyceryl-10 Didecanoate, Polyglyceryl-10 Diisostearate, Polyglyceryl-10 Dilaurate, Polyglyceryl-10 Dimyristate, Polyglyceryl-10 Dioleate, Polyglyceryl-10 Dipalmitate, Polyglyceryl-10 Distearate.

Thanks to the advantageous cosmetic and technical action, the second polyglycerol difatty acid ester C is preferably selected from the group of penta-, hexa- or heptaglycerol difatty acid esters, preferably from the group of hexaglycerol difatty acid esters.

Thanks to the cosmetic and technical action thereof, preferred polyglycerol difatty acid esters C are those from the group of hexaglycerol difatty acid esters of saturated or unsaturated fatty acids with 16 to 30 carbon atoms, preferably from the group of hexaglycerol difatty acid esters of saturated or unsaturated fatty acids with 16 to 24 carbon atoms and in particular from the group of hexaglycerol difatty acid esters of stearic acid.

More preferred polyglycerol difatty acid esters C are selected from the group of compounds with the INCI name PEG-6 Distearate.

The proportion by weight of the polyglycerol difatty acid ester C in the total weight of the compositions preferably amounts to from 0.2 to 8.0 wt. %, preferably from 0.4 to 6.0 wt. % and in particular from 0.6 to 4.0 wt. %.

The fatty acid ester of the general formula (I) is a further essential component of the cosmetic compositions. Thanks to the cosmetic and technical advantages thereof, preferred fatty acid esters are those in which the residues R₁ and R₂ in the general formula (I) mutually independently denote an unsaturated alkyl residue with 16 to 26 carbon atoms, preferably an unsaturated alkyl residue with 18 to 24 carbon atoms.

More preferred fatty acid esters of the general formula (I) are from the group of compounds with the INCI name Jojoba Esters.

Preferably, the proportion by weight of the fatty acid ester of the general formula (I) in the total weight of the compositions amounts to 0.05 to 3.0 wt. %, preferably from 0.1 to 2.0 wt. % and in particular from 0.2 to 1.5 wt. %.

Thanks to the advantageous cosmetic action and the high chemical as well as physical storage stability, it is more preferred to use mixtures of polyglycerol difatty acid esters B, polyglycerol difatty acid esters C and fatty acid esters of the general formula (I) which include, relative to the total weight of the mixture
5.0 to 10 wt. % polyglycerol difatty acid esters B,
50 to 75 wt. % polyglycerol difatty acid esters C and
10 to 25 wt. % fatty acid ester of the general formula (I).

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

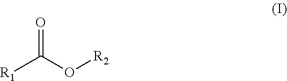

the residues R₁ and R₂ mutually independently denoting an unsaturated alkyl residue with 16 to 26 carbon atoms, and
e) at least one organic UV filter.

In a preferred embodiment, the cosmetic compositions furthermore include as optional component a linear fatty alcohol, preferably a linear fatty alcohol with 14 to 18 carbon atoms, in particular a linear fatty alcohol with 16 carbon atoms.

Relative to the total weight of the compositions, the proportion by weight of the linear fatty alcohol preferably amounts to 0.01 to 5.0 wt. %, preferably from 0.05 to 3.0 wt. % and in particular from 0.1 to 1.0 wt. %.

The compositions include at least one organic UV filter as cosmetic active substance.

Through the addition of a UV filter, both the agents themselves and the treated fibres may be protected from the harmful effects of UV radiation. At least one UV filter is accordingly preferably added to the agent. Suitable UV filters are not subject to any general restrictions with regard to structure and physical properties. Rather, any UV filters usable in the field of cosmetics whose absorption maximum is in the UVA (315-400 nm), the UVB (280-315 nm) or the UVC (<280 nm) range are suitable. UV filters with an absorption maximum in the UVB range, in particular in the range from approximately 280 to approximately 300 nm, are more preferred.

Examples of UV filters usable according to the invention are 4-aminobenzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts thereof, (Phenylbenzimidazole Sulfonic Acid; Parsol®HS; Neo Heliopan® Hydro), 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)-propane-1,3-dione (Butyl Methoxydibenzoylmethane; Parsol®1789, Eusolex®9020), α-(2-oxoborn-3-ylidene)-toluene-4-sulfonic acid and the salts thereof, ethoxylated 4-aminobenzoic acid ethyl ester (PEG-25 PABA; Uvinul®P 25), 4-dimethylaminobenzoic acid 2-ethylhexyl ester (Octyl Dimethyl PABA; Uvasorb®DMO, Escalol®507, Eusolex®6007), salicylic acid 2-ethylhexyl ester (Octyl Salicylate; Escalol®587, Neo Heliopan®OS, Uvinul®O18), 4-methoxycinnamic acid isopentyl ester (Isoamyl p-Methoxycinnamate; Neo Heliopan®E 1000), 4-methoxycinnamic acid 2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene Camphor; Parsol®5000, Eusolex®6300), 3-benzylidenecamphor (3-Benzylidene Camphor), 4-isopropylbenzyl salicylate 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide, 2,4-dihydroxybenzophenone (Benzophenone-1; Uvasorb®20 H, Uvinul®400), 1,1′-diphenylacrylonitrile acid 2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzo-phenone-5-sodium sulfonate, 2-cyano-3,3-diphenylacrylic acid-2′-ethylhexyl ester and phenylene-1,4-bis-(2-benzimidazole)-3,3′-5,5′-tetrasulfonic acid-bis-sodium salt (Disodium Phenyl Dibenzimidazole Tetrasulfonate, Neo Heliopan® AP).

UV filters which are preferred according to the invention are substituted dibenzoylmethanes, diphenylacrylic acid esters, salicylic acid esters and benzimidazoles.

More preferred organic UV filters are
3,3,5-trimethylcyclohexyl salicylate,
2-phenylbenzimidazole-5-sulfonic acid and the salts thereof,
1-(4-tert.-butylphenyl)-3-(4-methoxyphenyl)propane-1,3-dione,
1,1′-diphenylacrylonitrile acid 2-ethylhexyl ester, preferably in combination with ethylenediaminetetraacetic acid and the salts thereof,
phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine.

It goes without saying that combinations of organic UV filters may also be used in the cosmetic compositions according to the invention. Preferred combinations of UV filters here in particular include the UV filters previously described as more preferred. Thanks to their excellent physical stability, the cosmetic compositions according to the invention are particularly well suited for formulating a plurality of organic UV filters.

It is more preferred for the organic UV filter to include at least two, preferably at least three and in particular at least four organic UV filters from the group 3,3,5-trimethylcyclohexyl salicylate, 2-phenylbenzimidazole-5-sulfonic acid and the salts thereof, 1-(4-tert.-butylphenyl)-3-(4-methoxyphenyl)propane-1,3-dione, 1,1′-diphenyl-acrylonitrile acid 2-ethylhexyl ester and phenylene-1,4-bis-(2-benzimidazole)-3,3′-5,5′-tetrasulfonic acid and the salts thereof.

The proportion by weight of the organic UV filters in the total weight of the compositions preferably amounts to from 1.0 to 35 wt. %, preferably from 2.0 to 30 wt. % and in particular from 5.0 to 25 wt. %.

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

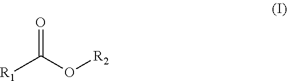

the residues R₁ and R₂ mutually independently denoting an unsaturated alkyl residue with 16 to 26 carbon atoms, and
e) 1.0 to 35 wt. % of at least one organic UV filter from the group
   3,3,5-trimethylcyclohexyl salicylate,
   2-phenylbenzimidazole-5-sulfonic acid and the salts thereof,
   1-(4-tert.-butylphenyl)-3-(4-methoxyphenyl)propane-1,3-dione,
   1,1′-diphenylacrylonitrile acid 2-ethylhexyl ester, preferably in combination with ethylenediaminetetraacetic acid and the salts thereof,
   phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine.

The compositions according to the invention include a linear alkane with 11 to 18 carbon atom as a further preferred optional component. Preferred linear alkanes are those which include 11 to 13 carbon atoms, it being more preferred for the linear alkane to include 11 and/or 13 carbon atoms.

The use of mixtures of undecane and tridecane is more preferred. The use of mixtures of undecane and tridecane is more preferred, in which the two linear alkanes are present in a weight ratio of 4:1 to 1:1, in particular of 3:1 to 1:1. Corresponding mixtures of substances are for example obtainable under the trade name Cetiol Ultimate.

Not only does the addition of the linear alkanes improve the storage stability of the cosmetic compositions, but addition of the above-described alkanes also improves the solubility of oil-soluble UV filters, improves the spreadability of the composition on the skin, reduces the tackiness of the composition and improves skin feel once the composition has been applied to the skin.

Cosmetic compositions in which the linear alkane is present, relative to the total weight of the compositions, in quantities of 0.1 to 10 wt. %, preferably of 0.2 to 7.0 wt. % and in particular of 0.5 to 5.0 wt. % are preferred.

In summary, preferred cosmetic compositions are those in the form of an oil-in-water emulsion including:
a) 0.01 to 6.0 wt. % of at least one monoglycerol fatty acid ester A from the group of mono-, di- or triesters of glycerol with behenic acid,
b) 0.05 to 3.0 wt. % of at least one first polyglycerol difatty acid ester B from the group of di-, tri- or tetraglycerol difatty acid esters of saturated or unsaturated fatty acids with 16 to 30 carbon atoms,
c) 0.2 to 8.0 wt. % of at least one second polyglycerol difatty acid ester C which differs from the first polyglycerol difatty acid ester from the group of penta-, hexa- or heptaglycerol difatty acid esters of saturated or unsaturated fatty acids with 16 to 30 carbon atoms,
d) 0.05 to 3.0 wt. % of at least one fatty acid ester of the general formula (I)

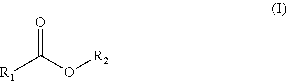

the residues R₁ and R₂ mutually independently denoting an unsaturated alkyl residue with 16 to 26 carbon atoms, and
e) 1.0 to 35 wt. % of at least one organic UV filter from the group
   3,3,5-trimethylcyclohexyl salicylate,
   2-phenylbenzimidazole-5-sulfonic acid and the salts thereof,
   1-(4-tert.-butylphenyl)-3-(4-methoxyphenyl)propane-1,3-dione,
   1,1′-diphenylacrylonitrile acid 2-ethylhexyl ester, preferably in combination with ethylenediaminetetraacetic acid and the salts thereof,
   phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine
f) 0.2 to 7.0 wt. % of a mixture of undecane and tridecane.

In order to establish a suitable viscosity and to further improve storage stability, preferred cosmetic compositions furthermore contain
at least one hydrophobically modified (meth)acrylic acid copolymer and/or
at least one copolymer of 2-methyl-2[(1-oxo-2-propenyl)amino]-1-propanesulfonic acid.
   It is more preferred to use a combination of both of the above-stated polymers.

The hydrophobically modified (meth)acrylic acid copolymers used are preferably copolymers which are attributable to
at least one first monomer from the group of unsaturated carboxylic acids and unsaturated carboxylic acid esters, and
at least one second monomer from the group of unsaturated hydrophobically modified monomers.

Preferred copolymers are based on at least one first monomer from the group acrylic acid, methacrylic acid, C₁-C₆ alkyl acrylic acid esters and C₁-C₆ alkyl methacrylic acid esters. The acrylic acid esters and methacrylic acid esters are preferably esters of the respective acids with non-tertiary alkyl alcohols having alkyl residues with 1 to 12 carbon atoms, in particular 2 to 4 carbon atoms. Suitable monomers which may be mentioned by way of example are ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate.

The group of hydrophobically modified second monomers denotes monomers which have a hydrophobic substructure. Preferred monomers may in turn be attributable to the following two structural units:
an unsaturated acid, preferably acrylic acid, methacrylic acid or itaconic acid;
a C₈₋₄₀ alkyl chain, preferably a C₁₀₋₃₀ alkyl chain.
   These two substructures may optionally be supplemented by a third structural unit from the group of polyoxyalkylene groups, preferably of polyethylene glycol groups, polypropylene glycol groups or polyethylene glycol/polypropylene glycol groups.

C₁₀₋₃₀ alkyl acrylates, C₁₀₋₃₀ alkyl PEG acrylates, C₁₀₋₃₀ alkyl PEG methacrylates or C₁₀₋₃₀ alkyl PEG itaconates are, for example, used as the second monomer. Preferred monomers are selected from C₁₀₋₃₀ alkyl acrylates, C₁₀₋₃₀ alkyl PEG 20-25 acrylates, C10-30 alkyl PEG 20-25 methacrylates or C₁₀₋₃₀ alkyl PEG 20-25 itaconates. More preferred monomers are selected from the group of C₁₀₋₃₀ alkyl acrylates, Steareth-20 methacrylates, Beheneth-25 methacrylates, Steareth-20 itaconates, Ceteth-20 itaconates, Palmeth-25 acrylates or C₁₀₋₃₀ alkyl PEG-20 itaconates.

In summary, preferred hydrophobically modified (meth)acrylic acid copolymers are those which are attributable to
at least one monomer from the group acrylic acid, methacrylic acid, C₁-C₆ alkyl acrylic acid esters, C₁-C₆ alkyl methacrylic acid esters, and
at least one monomer from the group of C₁₀₋₃₀ alkyl acrylates, C₁₀₋₃₀ alkyl methacrylates, C₁₀₋₃₀ alkyl PEG-acrylates, C₁₀₋₃₀ alkyl PEG methacrylates or C₁₀₋₃₀ alkyl PEG itaconates, preferably from the group of C₁₀₋₃₀ alkyl acrylates.

More preferred hydrophobically modified (meth)acrylic acid copolymers are selected from the group of compounds with the INCI name Acrylates/C₁₀₋₃₀ Alkyl Acrylate Crosspolymer, as are for example obtainable under the trade names Carbopol ETD 2020, Ultrez 21 or Pemulen TR1.

The proportion by weight of the hydrophobically modified (meth)acrylic acid copolymer in the total weight of the composition preferably amounts to from 0.05 to 2.0 wt. %, preferably from 0.1 to 1.2 wt. % and in particular 0.2 to 0.8 wt. %.

Preferably used copolymers of 2-methyl-2[(1-oxo-2-propenyl)amino]-1-propanesulfonic acid are those copolymers which are attributable to
2-methyl-2[(1-oxo-2-propenyl)amino]-1-propanesulfonic acid and
vinylpyrrolidone.
   These copolymers are preferably crosslinked.

Crosslinked copolymers of vinylpyrrolidone and 2-methyl-2[(1-oxo-2-propenyl)-amino]-1-propanesulfonic acid are for example obtainable by copolymerizing the two monomers in the presence of ammonium hydroxide and a crosslinking agent. Crosslinking may proceed with the assistance of olefinically polyunsaturated compounds, for example divinylbenzene, tetraallyloxyethane, methylenebisacrylamide, diallyl ether, polyallyl polyglyceryl ether or allyl ethers of sugars or sugar derivatives such as erythritol, pentaerythritol, arabitol, mannitol, sorbitol, sucrose or glucose. Methylenebisacrylamide is a preferred crosslinking agent.

One particularly suitable copolymer is the commercial product Aristoflex® AVC (from Clariant) which, as the ammonium salt, assumes the form of a white powder.

The copolymer of 2-methyl-2[(1-oxo-2-propenyl)amino]-1-propanesulfonic acid is preferably present in the compositions according to the invention in quantities of 0.05 to 3.0 wt. %, preferably of 0.1 to 2.0 wt. % and in particular of 0.2 to 1.5 wt. %.

### Summary Table

The following tables show the composition of some preferred cosmetic compositions (values stated in wt. % relative to the total weight of the cosmetic composition unless otherwise stated).

| | Formula 1 | Formula 2 | Formula 3 | Formula 4 |
|---|---|---|---|---|
| Monoglycerol fatty | 0.01 to 6.0 | 0.1 to 4.0 | 0.1 to 4.0 | 0.5 to 2.0 |
| acid ester A | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 5 | Formula 6 | Formula 7 | Formula 8 |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 11 | Formula 12 | Formula 13 | Formula 14 |
|---|---|---|---|---|
| Monoglycerol fatty acid ester A | 0.01 to 6.0 | 0.1 to 4.0 | 0.1 to 4.0 | 0.5 to 2.0 |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 15 | Formula 16 | Formula 17 | Formula 18 |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I) | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 21 | Formula 22 | Formula 23 | Formula 24 |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 25 | Formula 26 | Formula 27 | Formula 28 |
|---|---|---|---|---|
| Monoglycerol fatty acid ester A | 0.01 to 6.0 | 0.1 to 4.0 | 0.1 to 4.0 | 0.5 to 2.0 |
| Polyglycerol difatty acid ester B | 0.05 to 3.0 | 0.05 to 3.0 | 0.1 to 2.0 | 0.2 to 1.5 |
| Polyglycerol difatty acid ester C | 0.2 to 8.0 | 0.2 to 8.0 | 0.4 to 6.0 | 0.6 to 4.0 |
| Fatty acid ester of the general | 0.05 to 3.0 | 0.1 to 2.0 | 0.1 to 2.0 | 0.2 to 1.5 |
| formula (I) | | | | |
| Organic UV filter¹⁾ | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 31 | Formula 32 | Formula 33 | Formula 34 |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 35 | Formula 36 | Formula 37 | Formula 38 |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of unsaturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated C₁₆₋₂₆ alkyl residue | | | | |
| Organic UV filter¹⁾ | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | | | Formula | Formula |
|---|---|---|---|---|
| | Formula 41 | Formula 42 | 43 | 44 |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | | | Formula | Formula |
|---|---|---|---|---|
| | Formula 45 | Formula 46 | 47 | 48 |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Organic UV filter¹⁾ | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 1a | Formula 2a | Formula 3a | Formula 4a |
|---|---|---|---|---|
| Monoglycerol fatty | 0.01 to 6.0 | 0.1 to 4.0 | 0.1 to 4.0 | 0.5 to 2.0 |
| acid ester A | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of the | 0.05 to 3.0 | 0.1 to 2.0 | 0.1 to 2.0 | 0.2 to 1.5 |
| general formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 5a | Formula 6a | Formula 7a | Formula 8a |
|---|---|---|---|---|
| Mono-, di- or triester | 0.01 to 6.0 | 0.1 to 4.0 | 0.1 to 4.0 | 0.5 to 2.0 |
| of glycerol with | | | | |
| behenic acid | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of | 0.05 to 3.0 | 0.1 to 2.0 | 0.1 to 2.0 | 0.2 to 1.5 |
| the general | | | | |
| formula (I) | | | | |
| Linear C₁₄₋₁₈ fatty | 0.01 to 5.0 | 0.05 to 3.0 | 0.05 to 3.0 | 0.1 to 1.0 |
| alcohol | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 11a | Formula 12a | Formula 13a | Formula 14a |
|---|---|---|---|---|
| Monoglycerol fatty acid ester | 0.01 to 6.0 | 0.1 to 4.0 | 0.1 to 4.0 | 0.5 to 2.0 |
| A | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Polyglycerol difatty acid ester | 0.2 to 8.0 | 0.2 to 8.0 | 0.4 to 6.0 | 0.6 to 4.0 |
| C | | | | |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 15a | Formula 16a | Formula 17a | Formula 18a |
|---|---|---|---|---|
| Monoglycerol fatty acid ester | 0.01 to 6.0 | 0.1 to 4.0 | 0.1 to 4.0 | 0.5 to 2.0 |
| A | | | | |
| Polyglycerol difatty acid ester | 0.05 to 3.0 | 0.05 to 3.0 | 0.1 to 2.0 | 0.2 to 1.5 |
| B | | | | |
| Penta-, hexa- or heptaglycerol | 0.2 to 8.0 | 0.2 to 8.0 | 0.4 to 6.0 | 0.6 to 4.0 |
| difatty acid ester of unsaturated | | | | |
| C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 21a | Formula 22a | Formula 23a | Formula 24a |
|---|---|---|---|---|
| Monoglycerol fatty acid ester | 0.01 to 6.0 | 0.1 to 4.0 | 0.1 to 4.0 | 0.5 to 2.0 |
| A | | | | |
| Polyglycerol difatty acid ester | 0.05 to 3.0 | 0.05 to 3.0 | 0.1 to 2.0 | 0.2 to 1.5 |
| B | | | | |
| Polyglycerol difatty acid ester | 0.2 to 8.0 | 0.2 to 8.0 | 0.4 to 6.0 | 0.6 to 4.0 |
| C | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 25a | Formula 26a | Formula 27a | Formula 28a |
|---|---|---|---|---|
| Monoglycerol fatty acid ester | 0.01 to 6.0 | 0.1 to 4.0 | 0.1 to 4.0 | 0.5 to 2.0 |
| A | | | | |
| Polyglycerol difatty acid ester | 0.05 to 3.0 | 0.05 to 3.0 | 0.1 to 2.0 | 0.2 to 1.5 |
| B | | | | |
| Polyglycerol difatty acid ester | 0.2 to 8.0 | 0.2 to 8.0 | 0.4 to 6.0 | 0.6 to 4.0 |
| C | | | | |
| Fatty acid ester of the general | 0.05 to 3.0 | 0.1 to 2.0 | 0.1 to 2.0 | 0.2 to 1.5 |
| formula (I) | | | | |
| Linear C₁₄₋₁₈ fatty alcohol | 0.01 to 5.0 | 0.05 to 3.0 | 0.05 to 3.0 | 0.1 to 1.0 |
| Organic UV filter¹⁾ | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 31a | Formula 32a | Formula 33a | Formula 34a |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 35a | Formula 36a | Formula 37a | Formula 38a |
|---|---|---|---|---|
| Mono-, di- or triester of | 0.01 to 6.0 | 0.1 to 4.0 | 0.1 to 4.0 | 0.5 to 2.0 |
| glycerol with behenic acid | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₄₋₁₈ fatty alcohol | 0.01 to 5.0 | 0.05 to 3.0 | 0.05 to 3.0 | 0.1 to 1.0 |
| Organic UV filter¹⁾ | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 41a | 42a | 43a | 44a |
| Glyceryl Behenate | 0.01 to 6.0 | 0.1 to 4.0 | 0.1 to 4.0 | 0.5 to 2.0 |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 45a | 46a | 47a | 48a |
| Glyceryl | 0.01 to 6.0 | 0.1 to 4.0 | 0.1 to 4.0 | 0.5 to 2.0 |
| Behenate | | | | |
| PEG-3 Beeswax | 0.05 to 3.0 | 0.05 to 3.0 | 0.1 to 2.0 | 0.2 to 1.5 |
| PEG-6 Distearate | 0.2 to 8.0 | 0.2 to 8.0 | 0.4 to 6.0 | 0.6 to 4.0 |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

| | Formula 1c | Formula 2c | Formula 3c | Formula 4c |
|---|---|---|---|---|
| Monoglycerol fatty | 0.01 to 6.0 | 0.1 to 4.0 | 0.1 to 4.0 | 0.5 to 2.0 |
| acid ester A | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of | 0.05 to 3.0 | 0.1 to 2.0 | 0.1 to 2.0 | 0.2 to 1.5 |
| the general | | | | |
| formula (I) | | | | |
| Linear C₁₁₋₁₃ alkane | 0.1 to 10 | 0.2 to 7.0 | 0.2 to 7.0 | 0.5 to 5.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 5c | Formula 6c | Formula 7c | Formula 8c |
|---|---|---|---|---|
| Mono-, di- or triester | 0.01 to 6.0 | 0.1 to 4.0 | 0.1 to 4.0 | 0.5 to 2.0 |
| of glycerol with | | | | |
| behenic acid | | | | |
| Polyglycerol difatty | 0.05 to 3.0 | 0.05 to 3.0 | 0.1 to 2.0 | 0.2 to 1.5 |
| acid ester B | | | | |
| Polyglycerol difatty | 0.2 to 8.0 | 0.2 to 8.0 | 0.4 to 6.0 | 0.6 to 4.0 |
| acid ester C | | | | |
| Fatty acid ester of | 0.05 to 3.0 | 0.1 to 2.0 | 0.1 to 2.0 | 0.2 to 1.5 |
| the general formula | | | | |
| (I) | | | | |
| Linear C₁₁₋₁₃ alkane | 0.1 to 10 | 0.2 to 7.0 | 0.2 to 7.0 | 0.5 to 5.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 11c | Formula 12c | Formula 13c | Formula 14c |
|---|---|---|---|---|
| Monoglycerol fatty acid ester | 0.01 to 6.0 | 0.1 to 4.0 | 0.1 to 4.0 | 0.5 to 2.0 |
| A | | | | |
| Di-, tri- or tetraglycerol difatty | 0.05 to 3.0 | 0.05 to 3.0 | 0.1 to 2.0 | 0.2 to 1.5 |
| acid ester of saturated or | | | | |
| unsaturated C₁₆₋₁₈ fatty acids | | | | |
| Polyglycerol difatty acid ester | 0.2 to 8.0 | 0.2 to 8.0 | 0.4 to 6.0 | 0.6 to 4.0 |
| C | | | | |
| Fatty acid ester of the general | 0.05 to 3.0 | 0.1 to 2.0 | 0.1 to 2.0 | 0.2 to 1.5 |
| formula (I) | | | | |
| Linear C₁₁₋₁₃ alkane | 0.1 to 10 | 0.2 to 7.0 | 0.2 to 7.0 | 0.5 to 5.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 15c | Formula 16c | Formula 17c | Formula 18c |
|---|---|---|---|---|
| Monoglycerol fatty acid ester | 0.01 to 6.0 | 0.1 to 4.0 | 0.1 to 4.0 | 0.5 to 2.0 |
| A | | | | |
| Polyglycerol difatty acid ester | 0.05 to 3.0 | 0.05 to 3.0 | 0.1 to 2.0 | 0.2 to 1.5 |
| B | | | | |
| Penta-, hexa- or heptaglycerol | 0.2 to 8.0 | 0.2 to 8.0 | 0.4 to 6.0 | 0.6 to 4.0 |
| difatty acid ester of saturated | | | | |
| or unsaturated C₁₆₋₁₈ fatty acids | | | | |
| Fatty acid ester of the general | 0.05 to 3.0 | 0.1 to 2.0 | 0.1 to 2.0 | 0.2 to 1.5 |
| formula (I) | | | | |
| Linear C₁₁₋₁₃ alkane | 0.1 to 10 | 0.2 to 7.0 | 0.2 to 7.0 | 0.5 to 5.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 21c | Formula 22c | Formula 23c | Formula 24c |
|---|---|---|---|---|
| Monoglycerol fatty acid ester | 0.01 to 6.0 | 0.1 to 4.0 | 0.1 to 4.0 | 0.5 to 2.0 |
| A | | | | |
| Polyglycerol difatty acid ester | 0.05 to 3.0 | 0.05 to 3.0 | 0.1 to 2.0 | 0.2 to 1.5 |
| B | | | | |
| Polyglycerol difatty acid ester | 0.2 to 8.0 | 0.2 to 8.0 | 0.4 to 6.0 | 0.6 to 4.0 |
| C | | | | |
| Fatty acid ester of the general | 0.05 to 3.0 | 0.1 to 2.0 | 0.1 to 2.0 | 0.2 to 1.5 |
| formula (I), in which the | | | | |
| residues R₁ and R₂ mutually | | | | |
| independently denote an | | | | |
| unsaturated | | | | |
| C₁₆₋₂₆ alkyl residue | | | | |
| Linear C₁₁₋₁₃ alkane | 0.1 to 10 | 0.2 to 7.0 | 0.2 to 7.0 | 0.5 to 5.0 |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 25c | Formula 26c | Formula 27c | Formula 28c |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Polyglycerol | 0.2 to 8.0 | 0.2 to 8.0 | 0.4 to 6.0 | 0.6 to 4.0 |
| difatty acid | | | | |
| ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Linear | 0.1 to 10 | 0.2 to 7.0 | 0.2 to 7.0 | 0.5 to 5.0 |
| C₁₁₋₁₃ alkane | | | | |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 31c | 32c | 33c | 34c |
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Di-, tri- or | 0.05 to 3.0 | 0.05 to 3.0 | 0.1 to 2.0 | 0.2 to 1.5 |
| tetraglycerol | | | | |
| difatty | | | | |
| acid ester of | | | | |
| saturated or | | | | |
| unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), in | | | | |
| which the | | | | |
| residues R₁ and | | | | |
| R₂ mutually | | | | |
| independently | | | | |
| denote an | | | | |
| unsaturated C₁₆₋₂₆ | | | | |
| alkyl residue | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 35c | Formula 36c | Formula 37c | Formula 38c |
|---|---|---|---|---|
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Di-, tri- or | 0.05 to 3.0 | 0.05 to 3.0 | 0.1 to 2.0 | 0.2 to 1.5 |
| tetraglycerol | | | | |
| difatty | | | | |
| acid ester of | | | | |
| saturated or | | | | |
| unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), in | | | | |
| which the | | | | |
| residues R₁ and | | | | |
| R₂ mutually | | | | |
| independently | | | | |
| denote an | | | | |
| unsaturated | | | | |
| C₁₆₋₂₆ alkyl | | | | |
| residue | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

| | Formula 41c | Formula 42c | Formula 43c | Formula 44c |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Undecane, | 0.1 to 10 | 0.2 to 7.0 | 0.2 to 7.0 | 0.5 to 5.0 |
| tridecane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 45c | Formula 46c | Formula 47c | Formula 48c |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Undecane, | 0.1 to 10 | 0.2 to 7.0 | 0.2 to 7.0 | 0.5 to 5.0 |
| tridecane | | | | |
| Organic | 1.0 to 35 | 2.0 to 30 | 2.0 to 30 | 5.0 to 25 |
| UV filter¹⁾ | | | | |
| Water | 30 to 82 | 30 to 82 | 35 to 75 | 45 to 60 |
| Misc. | ad 100 | ad 100 | ad 100 | ad 100 |

| | Formula 1d | Formula 2d | Formula 3d | Formula 4d |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Polyglycerol | 0.2 to 8.0 | 0.2 to 8.0 | 0.4 to 6.0 | 0.6 to 4.0 |
| difatty acid | | | | |
| ester C | | | | |
| Fatty acid | 0.05 to 3.0 | 0.1 to 2.0 | 0.1 to 2.0 | 0.2 to 1.5 |
| ester of the | | | | |
| general | | | | |
| formula (I) | | | | |
| Linear C₁₄₋₁₈ | 0.01 to 5.0 | 0.05 to 3.0 | 0.05 to 3.0 | 0.1 to 1.0 |
| fatty | | | | |
| alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 5d | Formula 6d | Formula 7d | Formula 8d |
|---|---|---|---|---|
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Polyglycerol | 0.05 to 3.0 | 0.05 to 3.0 | 0.1 to 2.0 | 0.2 to 1.5 |
| difatty | | | | |
| acid ester B | | | | |
| Polyglycerol | 0.2 to 8.0 | 0.2 to 8.0 | 0.4 to 6.0 | 0.6 to 4.0 |
| difatty | | | | |
| acid ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 11d | Formula 12d | Formula 13d | Formula 14d |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Di-, tri- or | 0.05 to 3.0 | 0.05 to 3.0 | 0.1 to 2.0 | 0.2 to 1.5 |
| tetraglycerol | | | | |
| difatty acid | | | | |
| ester | | | | |
| of saturated | | | | |
| or unsaturated | | | | |
| C₁₆₋₁₈ fatty acids | | | | |
| Polyglycerol | 0.2 to 8.0 | 0.2 to 8.0 | 0.4 to 6.0 | 0.6 to 4.0 |
| difatty | | | | |
| acid ester C | | | | |
| Fatty acid ester | 0.05 to 3.0 | 0.1 to 2.0 | 0.1 to 2.0 | 0.2 to 1.5 |
| of the general | | | | |
| formula (I) | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 15d | 16d | 17d | 18d |
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Penta-, hexa- or | 0.2 to 8.0 | 0.2 to 8.0 | 0.4 to 6.0 | 0.6 to 4.0 |
| heptaglycerol | | | | |
| difatty acid | | | | |
| ester of | | | | |
| saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I) | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 21d | Formula 22d | Formula 23d | Formula 24d |
|---|---|---|---|---|
| Monoglycerol | 0.01 to 6.0 | 0.1 to 4.0 | 0.1 to 4.0 | 0.5 to 2.0 |
| fatty | | | | |
| acid ester A | | | | |
| Polyglycerol | 0.05 to 3.0 | 0.05 to 3.0 | 0.1 to 2.0 | 0.2 to 1.5 |
| difatty | | | | |
| acid ester B | | | | |
| Polyglycerol | 0.2 to 8.0 | 0.2 to 8.0 | 0.4 to 6.0 | 0.6 to 4.0 |
| difatty | | | | |
| acid ester C | | | | |
| Fatty acid | 0.05 to 3.0 | 0.1 to 2.0 | 0.1 to 2.0 | 0.2 to 1.5 |
| ester of | | | | |
| the general | | | | |
| formula (I), in | | | | |
| which the | | | | |
| residues | | | | |
| R₁ and R₂ | | | | |
| mutually | | | | |
| independently | | | | |
| denote an | | | | |
| unsaturated | | | | |
| C₁₆₋₂₆ alkyl | | | | |
| residue | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 25d | Formula 26d | Formula 27d | Formula 28d |
|---|---|---|---|---|
| Monoglycerol | 0.01 to 6.0 | 0.1 to 4.0 | 0.1 to 4.0 | 0.5 to 2.0 |
| fatty acid | | | | |
| ester A | | | | |
| Polyglycerol | 0.05 to 3.0 | 0.05 to 3.0 | 0.1 to 2.0 | 0.2 to 1.5 |
| difatty acid | | | | |
| ester B | | | | |
| Polyglycerol | 0.2 to 8.0 | 0.2 to 8.0 | 0.4 to 6.0 | 0.6 to 4.0 |
| difatty acid ester | | | | |
| Fatty acid ester | 0.05 to 3.0 | 0.1 to 2.0 | 0.1 to 2.0 | 0.2 to 1.5 |
| of the general | | | | |
| formula (I) | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

| | Formula | Formula | Formula | Formula |
|---|---|---|---|---|
| | 31d | 32d | 33d | 34d |
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Di-, tri- or | 0.05 to 3.0 | 0.05 to 3.0 | 0.1 to 2.0 | 0.2 to 1.5 |
| tetraglycerol | | | | |
| difatty acid | | | | |
| ester of | | | | |
| saturated or | | | | |
| unsaturated C₁₆₋₁₈ | | | | |
| fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), in | | | | |
| which the | | | | |
| residues R₁ and | | | | |
| R₂ mutually | | | | |
| independently | | | | |
| denote an | | | | |
| unsaturated | | | | |
| C₁₆₋₂₆ alkyl | | | | |
| residue | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV filter | 1.0 to 35 | 2.0 to 30 | 2.0 to 30 | 5.0 to 25 |
| Water | 30 to 82 | 30 to 82 | 35 to 75 | 45 to 60 |
| Misc. | ad 100 | ad 100 | ad 100 | ad 100 |

| | Formula 35d | Formula 36d | Formula 37d | Formula 38d |
|---|---|---|---|---|
| Mono-, di- or | 0.01 to 6.0 | 0.1 to 4.0 | 0.1 to 4.0 | 0.5 to 2.0 |
| triester of | | | | |
| glycerol with | | | | |
| behenic acid | | | | |
| Di-, tri- or | 0.05 to 3.0 | 0.05 to 3.0 | 0.1 to 2.0 | 0.2 to 1.5 |
| tetraglycerol | | | | |
| difatty | | | | |
| acid ester of | | | | |
| saturated or | | | | |
| unsaturated | | | | |
| C₁₆₋₁₈ fatty acids | | | | |
| Penta-, hexa- or | 0.2 to 8.0 | 0.2 to 8.0 | 0.4 to 6.0 | 0.6 to 4.0 |
| heptaglycerol | | | | |
| difatty acid ester | | | | |
| of saturated | | | | |
| or unsaturated | | | | |
| C₁₆₋₁₈ fatty acids | | | | |
| Fatty acid ester | 0.05 to 3.0 | 0.1 to 2.0 | 0.1 to 2.0 | 0.2 to 1.5 |
| of the general | | | | |
| formula (I), in | | | | |
| which the | | | | |
| residues R₁ and | | | | |
| R₂ mutually | | | | |
| independently | | | | |
| denote an | | | | |
| unsaturated | | | | |
| C₁₆₋₂₆ alkyl | | | | |
| residue | | | | |
| Linear C₁₄₋₁₈ | 0.01 to 5.0 | 0.05 to 3.0 | 0.05 to 3.0 | 0.1 to 1.0 |
| fatty alcohol | | | | |
| Linear C₁₁₋₁₃ | 0.1 to 10 | 0.2 to 7.0 | 0.2 to 7.0 | 0.5 to 5.0 |
| alkane | | | | |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

| | Formula 41d | Formula 42d | Formula 43d | Formula 44d |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Undecane, | 0.1 to 10 | 0.2 to 7.0 | 0.2 to 7.0 | 0.5 to 5.0 |
| tridecane | | | | |
| Organic UV | 1.0 to 35 | 2.0 to 30 | 2.0 to 30 | 5.0 to 25 |
| filter | | | | |
| Water | 30 to 82 | 30 to 82 | 35 to 75 | 45 to 60 |
| Misc. | ad 100 | ad 100 | ad 100 | ad 100 |

| | Formula 45d | Formula 46d | Formula 47d | Formula 48d |
|---|---|---|---|---|
| Glyceryl | 0.01 to 6.0 | 0.1 to 4.0 | 0.1 to 4.0 | 0.5 to 2.0 |
| Behenate | | | | |
| PEG-3 | 0.05 to 3.0 | 0.05 to 3.0 | 0.1 to 2.0 | 0.2 to 1.5 |
| Beeswax | | | | |
| PEG-6 | 0.2 to 8.0 | 0.2 to 8.0 | 0.4 to 6.0 | 0.6 to 4.0 |
| Distearate | | | | |
| Jojoba Esters | 0.05 to 3.0 | 0.1 to 2.0 | 0.1 to 2.0 | 0.2 to 1.5 |
| Cetyl Alcohol | 0.01 to 5.0 | 0.05 to 3.0 | 0.05 to 3.0 | 0.1 to 1.0 |
| Undecane, | 0.1 to 10 | 0.2 to 7.0 | 0.2 to 7.0 | 0.5 to 5.0 |
| tridecane | | | | |
| Organic UV | 1.0 to 35 | 2.0 to 30 | 2.0 to 30 | 5.0 to 25 |
| filter¹⁾ | | | | |
| Water | 30 to 82 | 30 to 82 | 35 to 75 | 45 to 60 |
| Misc. | ad 100 | ad 100 | ad 100 | ad 100 |

1) the organic UV filter is selected from the group that includes
   3,3,5-trimethylcyclohexyl salicylate,
   2-phenylbenzimidazole-5-sulfonic acid and the salts thereof,
   1-(4-tert.-butylphenyl)-3-(4-methoxyphenyl)propane-1,3-dione,
   1,1′-diphenylacrylonitrile acid 2-ethylhexyl ester, preferably in combination with ethylenediaminetetraacetic acid and the salts thereof, and
   phenylene-1,4-bis-(2-benzimidazole)-3,3′-5,5′-tetrasulfonic acid and the salts thereof, preferably in combination with an amino acid, preferably in combination with arginine, histidine, ornithine or lysine, in particular in combination with arginine.

As explained above, a combination of components a), b), c) and d) is particularly suitable for improving both the physical and the chemical stability of cosmetic UV screening agents. The present application also provides the use of a combination that includes
a) at least one monoglycerol fatty acid ester A,
b) at least one first polyglycerol difatty acid ester B,
c) at least one second polyglycerol difatty acid ester C which differs from the first polyglycerol difatty acid ester,
d) at least one fatty acid ester of the general formula (I)

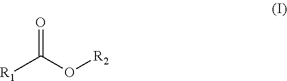

the residues R₁ and R₂ mutually independently denoting a saturated or unsaturated alkyl residue with 16 to 26 carbon atoms, for
improving the storage stability of a cosmetic composition including at least one UV filter;
improving the UV-protective action of a cosmetic composition including at least one UV filter;
improving the absorbability of a cosmetic composition including at least one UV filter;
improving the spreadability and reducing the tackiness of a cosmetic composition including at least one UV filter.

The present invention thirdly and finally provides a method for cosmetic skin care, in which a cosmetic composition according to the invention is applied onto the skin, in particular the facial skin.

The statements made regarding the cosmetic agents according to the invention apply mutatis mutandis to the use according to the invention and to the cosmetic method according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition in the form of an oil-in-water emulsion including:
a) at least one monoglycerol fatty acid ester A,
b) at least one first polyglycerol difatty acid ester B,
c) at least one second polyglycerol difatty acid ester C which differs from the first polyglycerol difatty acid ester B,
d) at least one fatty acid ester of the general formula (I) 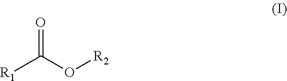
the residues R₁ and R₂ mutually independently denoting a saturated or unsaturated alkyl residue with 16 to 26 carbon atoms,
e) at least one organic UV filter.

2. The cosmetic composition according to claim 1, wherein the monoglycerol fatty acid ester A is present, relative to the total weight of the compositions, in quantities of 0.01 to 6.0 wt. %.

3. The cosmetic composition according to claim 1, wherein the first polyglycerol difatty acid ester B is present, relative to the total weight of the compositions, in quantities of 0.05 to 3.0 wt. %.

4. The cosmetic composition according to claim 1, wherein the second polyglycerol difatty acid ester C is present, relative to the total weight of the compositions, in quantities of 0.2 to 8.0 wt. %.

5. The cosmetic composition according to claim 1, wherein the fatty acid ester of the general formula (I) is present, relative to the total weight of the compositions, in quantities of 0.05 to 3.0 wt. %.

6. The cosmetic composition according to claim 1, wherein the UV filters are present, relative to the total weight of the compositions, in quantities of 1.0 to 35 wt. %.

7. The cosmetic composition according to claim 1, further including
f) a linear fatty alcohol, preferably a linear fatty alcohol with 14 to 18 carbon atoms and in particular a linear fatty alcohol with 16 carbon atoms.

8. The cosmetic composition according to claim 1, further including
g) a linear alkane with 11 to 18 carbon atoms.

9. A method for cosmetic skin care, including applying onto the skin the cosmetic composition according to claim 1.
